(19) United States
(12) Patent Application Publication
(10) Pub. No.: US 2019/0381337 A1
(43) Pub. Date: Dec. 19, 2019

(54) BORON NEUTRON CAPTURE THERAPY SYSTEM

(71) Applicant: NEUBORON MEDTECH LTD., Nanjing (CN)

(72) Inventor: Yuan-Hao LIU, Nanjing (CN)

(21) Appl. No.: 16/459,908

(22) Filed: Jul. 2, 2019

Related U.S. Application Data
(63) Continuation of application No. 16/134,018, filed on Sep. 18, 2018.
Application No. 16/134,018 is a continuation of application No. PCT/CN2017/076946, filed on Mar. 16, 2017.

(30) Foreign Application Priority Data
Mar. 25, 2016 (CN) 201610180136.4
Mar. 25, 2016 (CN) 201610180591.4
Mar. 25, 2016 (CN) 201620241984.7

(51) Int. Cl. A61N 5/10; C07F 5/02

(52) U.S. Cl. CPC A61N 5/1067; A61P 35/04; C07F 5/02

(57) ABSTRACT

The present disclosure discloses a boron neutron capture therapy system comprising: a boron neutron capture therapy device and an α-amino acid-like boron trifluoride compound having a structure shown as formula (I) below:

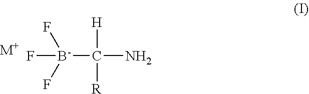

Wherein: R is selected from hydrogen, methyl, isopropyl, 1-methylpropyl, 2-methylpropyl, hydroxymethyl, 1-hydroxyethyl, benzyl or hydroxybenzyl; M is H or metal atom. The energy generated from the action of the neutron beam generated by the boron neutron capture therapy device on the α-amino acid-like boron trifluoride compound destroys tumor cell DNA. In another aspect, the present disclosure discloses a use of an α-amino acid-like boron trifluoride compound in the preparation of a medicament for tumor therapy.

### RELATED APPLICATION INFORMATION

This application is a continuation of U.S. patent application Ser. No. 16/134,018, filed on Sep. 18, 2018, which is a continuation of International Application No. PCT/CN2017/076946, filed on Mar. 16, 2017, which claims priority to Chinese Patent Application No. 201610180136.4, filed on Mar. 25, 2016, Chinese Patent Application No. 201610180591.4, filed on Mar. 25, 2016, and Chinese Patent Application No. 201620241984.7, filed on Mar. 25, 2016, the disclosures of which are hereby incorporated by reference.

### FIELD OF THE DISCLOSURE

One aspect of the present disclosure relates to a radioactive ray irradiation therapy system, in particular to a boron neutron capture therapy system. Another aspect of the present disclosure relates to the field of medicine, in particular to the field of tumor-related medicine, and more specifically, to use of α-amino acid-like boron trifluoride compound in the preparation of a medicament for tumor therapy.

### BACKGROUND OF THE DISCLOSURE

Along with the development of atomic science, radiation therapy, for example, cobalt 60, linear accelerator, electron beam and so on, has become one of the main means of cancer treatment. However, limited by the physical condition of the radioactive ray itself, conventional photon or electronic therapy brings injury to a large number of normal tissues in the beam pathway while killing tumor cells. In addition, due to the different levels of sensitivity of tumor cells to radioactive rays, traditional radiation therapy normally has poor therapeutic effects towards malignant tumors with higher radiation resistance (for example, glioblastoma multiforme, melanoma).

In order to reduce the radiation injury of normal tissues surrounding the tumor, the conception of target therapy in the chemotherapy is applied in radiation therapy now. In addition, towards tumor cells with high radiation resistance, radiation sources having high relative biological effectiveness (RBE) are also energetically developed at the moment, such as proton therapy, heavy particle therapy, neutron capture therapy and so on. Among those, neutron capture therapy, such as boron neutron capture therapy, is a combination of the two conceptions described above. By means of specific agglomeration of boron containing drugs towards tumor cells, in combination with precise neutron beam regulation, boron neutron capture therapy provides a cancer treatment option better than the traditional radioactive rays.

Boron neutron capture therapy (BNCT) uses the characteristic of boron (¹⁰B) containing drugs which have high capture cross-section with thermal neutrons, and produces two heavy charged particles ⁴He and ⁷Li, through ¹⁰B(n, α)⁷Li neutron capture nuclear fission reaction as follows:

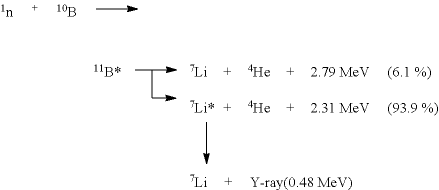

The average energy of those two charged particles is about 2.33 MeV The two charged particles have the characteristics of high Linear Energy Transfer (LET) and short range. The Linear Energy Transfer and the range of a particle are respectively 150 keV/μm, 8 μm. The Linear Energy Transfer and the range of heavy charged particle ⁷Li are respectively 175 keV/μm, 5 μm. The total range of two particles is approximately equivalent to the size of one cell, thus the radiation damage caused by them against living organism can be limited to the level of cells. When the boron containing drug selectively agglomerates in tumor cells, and matches with a proper neutron source, then it is possible to achieve the aim of killing tumor cells locally without harming normal tissues too much.

The effect of boron neutron capture therapy depends on the concentration of boron containing drug and the number of thermal neutrons in the location of tumor cells, so it is also known as binary cancer therapy; it can thus be seen that, in addition to the development of neutron source, the development of boron containing drugs plays an important role in the research of boron neutron capture therapy.

Tumors, especially malignant tumors, are diseases that seriously endanger human health in the world today. The mortality rates of tumors are second only to cardiovascular diseases, ranking second in the mortality rate of various types of diseases. In recent years, the incidence rates have shown a clear upward trend. According to the current incidence of cancer, the number of new cancer patients in the world will reach 15 million each year. Although the exact mechanisms of cancer developments are still unclear, most cancer patients are likely to survive if they can diagnose the cancer early and take early surgery, radiation or chemotherapy (or a combination of these methods).

A promising new form of high LET radiation cancer therapy is boron neutron capture therapy (BNCT). BNCT is a novel dual-targeted radiation therapy based on selective accumulation of boron, known as boron-10 or ¹⁰B, in tumors, followed by irradiation of tumors with thermalized neutrons. Thermalized neutrons strike boron-10, leading to nuclear fission (decay reaction). Nuclear fission reactions cause highly localized release of energy in the form of linear energy transfer (energy density, LET) radiation, which can kill cells more efficiently than low LET radiation, such as X-rays (higher relative biological effects).

In BNCT, when administered in a therapeutically effective amount, the boron-containing compound must be non-toxic or low toxic, and can selectively accumulate in tumor tissue. Although BPA has the advantage of low chemical toxicity, it accumulates in critical normal tissues at below-desired levels. In particular, the ratio of boron in the tumor relative to normal brain and tumor to blood is approximately 3:1. This low specificity limits the maximum dose of BPA to the tumor because the allowable dose for normal tissue is a limiting factor.

Therefore, there is a need to develop new compounds that have a longer retention time in tumors and selectively target and destroy tumor cells with minimal damage to normal tissues.

α-amino acids are the main components of proteins and are the most important amino acids in organisms. They play a very important role in the production of ATP and in the process of neurotransmission. In addition, α-amino acids are also key nutrients for the survival and proliferation of cancer cells. Substitution of —COOH in the α-amino acid with —BF₃ yields an α-amino acid-like boron trifluoride compound, which is an α-amino acid isoelectronic compound. Studies have shown that the pathway for the uptake of α-amino acid-like boron trifluoride by the cells is the same as α-amino acids, and they all adopt enzyme-mediated pathways, and both have the same transport protein. The α-amino acid-like boron trifluoride compounds have attracted our intense attention in the design of novel boron carrier compounds for BNCT, which have high stability, good targeting, and high enrichment in tumor cells. Compared to FDG, the uptake of these compounds by the inflammatory region is almost negligible. In addition, the α-amino acid-like boron trifluoride compounds are readily synthesized and are usually prepared by reacting the corresponding boronic acid ester with KHF₂ under acidic conditions.

In addition, using ¹⁸F-labeled α-amino acid-like boron trifluoride compounds in BNCT, boron concentrations and distributions in and around tumors and all tissues within the radiation therapy volume can be measured non-invasively, accurately and rapidly before and during irradiation. This diagnostic information enables boron neutron capture therapy to be performed faster, more accurately, and more safely by reducing the exposure of epithermal neutrons to tissue regions known to contain high levels of boron.

### SUMMARY

In order to achieve the improvement of the existing boron neutron capture therapy system, one aspect of the present disclosure provides a boron neutron capture therapy system including: a boron neutron capture therapy device and an α-amino acid-like boron trifluoride compound.

The α-amino acid-like boron trifluoride compound has a structure shown as formula (I):

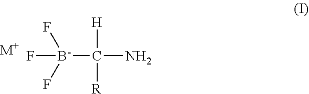

Wherein: R represents hydrogen, methyl, isopropyl, 1-methylpropyl, 2-methylpropyl, hydroxymethyl, 1-hydroxyethyl, benzyl or hydroxybenzyl; M represents H or metal atom.

The energy generated from the action of the neutron beam generated by the boron neutron capture therapy device on the α-amino acid-like boron trifluoride compound destroys tumor cell DNA.

Implementations of this aspect may include one or more of the following features.

BNCT is an ideal method for tumor therapy, which provides a new treatment for many tumors that cannot be treated with traditional methods.

Further, the tumor is a malignant tumor or metastatic tumor and is preferably glioma, recurrent head and neck tumor, malignant melanoma, breast cancer or metastatic hepatoma. Malignant neoplasms are often referred to as cancers, and they are collectively referred to as more than 100 related diseases. When cells in the body are mutated, it will continue to divide and not be controlled by the body, eventually developed into cancer. Malignant tumor cells can invade and destroy adjacent tissues and organs, and the cells can pass through the tumor and enter the blood or lymphatic system. This is how malignant tumors form new tumors from the primary site to other organs. It is called metastasis of malignant tumors.

Furthermore, the tumor is a brain tumor or a melanoma. A brain tumor is a tumor that grows in the brain, including primary brain tumors that occur in the brain parenchyma and secondary brain tumors that metastasize from the rest of the body to the brain. Melanoma, also known as malignant melanoma, is a highly malignant tumor that produces melanin. It occurs in the skin or in the mucous membranes close to the skin. It is also found in the pia mater and the choroid.

Preferably, the brain tumor is glioma. Neuroepithelioma-derived tumors are called gliomas and account for 40-50% of brain tumors. They are common intracranial malignancies.

The α-amino acid-like boron trifluoride compound plays an important role in the application of the boron neutron capture therapy system, which will be described in details below.

Preferably, M in the α-amino acid-like boron trifluoride compound represents potassium or sodium.

Preferably, the element B in the α-amino acid-like boron trifluoride compound is ¹⁰B.

In order to further increase the ¹⁰B content in the boron-containing drug, the purity of ¹⁰B in the α-amino acid-like boron trifluoride compound is ≥95%.

At least one F atom of the α-amino acid-like boron trifluoride compound is ¹⁸F, such that boron concentrations and distributions in and around tumors and all tissues within the radiation therapy volume can be measured non-invasively, accurately and rapidly before and during irradiation. This diagnostic information enables boron neutron capture therapy to be performed faster, more accurately, and more safely by reducing the exposure of epithermal neutrons to tissue regions known to contain high levels of boron.

Further, the boron neutron capture therapy device includes a neutron generator and a beam shaping assembly for adjusting a neutron beam energy spectrum generated by the neutron generator to an epithermal neutron energy zone.

The beam shaping assembly also plays an important role in improving the flux and quality of neutron sources. The beam shaping assembly includes a moderator adjacent to the neutron generator, a reflector surrounding the moderator, a thermal neutron absorber adjacent to the moderator, and a radiation shield arranged in the beam shaping assembly, the neutron generator generates neutrons by a nuclear reaction with the incident proton beam, the moderator moderates neutrons generated from the neutron generator to an epithermal neutron energy zone, the reflector directs the deviated neutrons back to increase the intensity of the epithermal neutron beam, the thermal neutron absorber is used to absorb thermal neutrons to avoid overdosing in superficial normal tissues during therapy, and the radiation shield is used to shield leaking neutrons and photons to reduce dose of the normal tissue not exposed to irradiation.

The boron neutron capture therapy device further includes a collimator disposed at the beam outlet for converging the epithermal neutrons.

Another aspect of the present disclosure is to provide novel use of α-amino acid-like boron trifluoride compound, and in particular to the use of α-amino acid-like boron trifluoride compound in the preparation of a medicament for tumor therapy.

The tumor therapy refers to boron neutron capture therapy of the tumor. The boron neutron capture therapy (BNCT) is a novel dual-targeted radiation therapy that destroys cancer cells through the nuclear fission of boron in the tumor cells. First of all, oral or intravenous injection of boron carrier agent with strong affinity for tumor cells, after the drug is enriched in tumor cells and irradiated with neutrons, the ¹⁰B atom undergoes a nuclear fission reaction, generating a particles and ⁷Li particles with high radiation energy and a small radiation range, which in turn selectively kills the tumor cells in which they are located. BNCT is an ideal method for tumor therapy, which provides a new therapy for many tumors that cannot be treated with traditional methods.

Further, the tumor is a malignant tumor or metastatic tumor and is preferably glioma, recurrent head and neck tumor, malignant melanoma, breast cancer or metastatic hepatoma. Malignant neoplasms are often referred to as cancers, and they are collectively referred to as more than 100 related diseases. When cells in the body are mutated, it will continue to divide and not be controlled by the body, eventually developed into cancer. Malignant tumor cells can invade and destroy adjacent tissues and organs, and the cells can pass through the tumor and enter the blood or lymphatic system. This is how malignant tumors form new tumors from the primary site to other organs. It is called metastasis of malignant tumors.

Further, the tumor is a brain tumor or a melanoma. A brain tumor is a tumor that grows in the brain, including primary brain tumors that occur in the brain parenchyma and secondary brain tumors that metastasize from the rest of the body to the brain. Melanoma, also known as malignant melanoma, is a highly malignant tumor that produces melanin. It occurs in the skin or in the mucous membranes close to the skin. It is also found in the pia mater and the choroid.

Preferably, the brain tumor is glioma. Neuroepithelioma-derived tumors are called gliomas and account for 40-50% of brain tumors. They are common intracranial malignancies.

The α-amino acid-like boron trifluoride compound has a structure shown as formula (I):

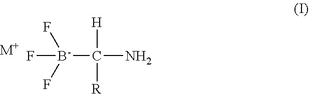

Wherein: R represents hydrogen, methyl, isopropyl, 1-methylpropyl, 2-methylpropyl, hydroxymethyl, 1-hydroxyethyl, benzyl or hydroxybenzyl.

The compound according to formula (I) can be obtained by the following preparation method, and the preparation route is as follows:

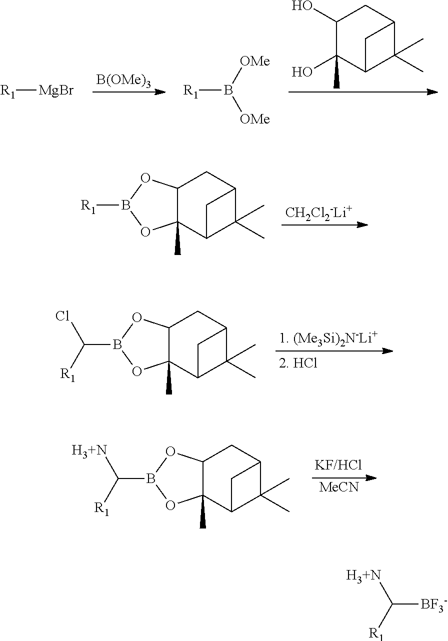

### DESCRIPTION OF THE FIGURES

FIG. 1 is a schematic plan view of an accelerator-based boron neutron capture therapy system.

FIG. 2 is a schematic plan view of a reactor-based boron neutron capture therapy system.

### DESCRIPTION OF THE DISCLOSURE

The following further describes the present disclosure in detail with reference to specific embodiments and the accompanying drawings so that those skilled in the art can implement the present disclosure with reference to the context of the specification. The purpose of the described embodiments is merely to illustrate and describe the current best mode of the present disclosure. The scope of the disclosure is not limited in any way by the embodiments described herein.

It will be understood that terms such as “having,” “including,” and “comprising” used herein do not exclude the presence or addition of one or more other components or combinations thereof.

The fast neutrons described herein are neutrons with an energy region greater than 40 keV, the epithermal neutron energy region is between 0.5 eV and 40 keV, and the thermal neutron energy region is less than 0.5 eV.

Neutron capture therapy (NCT) has been increasingly practiced as an effective cancer curing means in recent years, and BNCT is the most common. Neutrons for NCT may be supplied by nuclear reactors or accelerators. Take AB-BNCT for example, its principal components include, in general, an accelerator for accelerating charged particles (such as protons and deuterons), a target, a heat removal system and a beam shaping assembly. The accelerated charged particles interact with the metal target to produce the neutrons, and suitable nuclear reactions are always determined according to such characteristics as desired neutron yield and energy, available accelerated charged particle energy and current and materialization of the metal target, among which the most discussed two are ⁷Li (p, n) ⁷Be and ⁹Be (p, n) ⁹B and both are endothermic reaction. Their energy thresholds are 1.881 MeV and 2.055 MeV respectively. Epithermal neutrons at a keV energy level are considered ideal neutron sources for BNCT. Theoretically, bombardment with lithium target using protons with energy slightly higher than the thresholds may produce neutrons relatively low in energy, so the neutrons may be used clinically without many moderations. However, Li (lithium) and Be (beryllium) and protons of threshold energy exhibit not high action cross section. In order to produce sufficient neutron fluxes, high-energy protons are usually selected to trigger the nuclear reactions.

The target, considered perfect, is supposed to have the advantages of high neutron yield, a produced neutron energy distribution near the epithermal neutron energy range (see details thereinafter), little strong-penetration radiation, safety, low cost, easy accessibility, high temperature resistance etc. But in reality, no nuclear reactions may satisfy all requests. The target in these embodiments of the present disclosure is made of lithium. However, well known by those skilled in the art, the target materials may be made of other metals besides the above-mentioned.

Requirements for the heat removal system differ as the selected nuclear reactions. ⁷Li (p, n) ⁷Be asks for more than ⁹Be (p, n) ⁹B does because of low melting point and poor thermal conductivity coefficient of the metal (lithium) target. In these embodiments of the present disclosure is ⁷Li (p, n) ⁷Be.

No matter BNCT neutron sources are from the nuclear reactor or the nuclear reactions between the accelerator charged particles and the target, only mixed radiation fields are produced, that is, beams include neutrons and photons having energies from low to high. As for BNCT in the depth of tumors, except the epithermal neutrons, the more the residual quantity of radiation ray is, the higher the proportion of nonselective dose deposition in the normal tissue is. Therefore, radiation causing unnecessary dose should be lowered down as much as possible. Besides air beam quality factors, dose is calculated using a human head tissue prosthesis in order to understand dose distribution of the neutrons in the human body. The prosthesis beam quality factors are later used as design reference to the neutron beams, which is elaborated hereinafter.

The International Atomic Energy Agency (IAEA) has given five suggestions on the air beam quality factors for the clinical BNCT neutron sources. The suggestions may be used for differentiating the neutron sources and as reference for selecting neutron production pathways and designing the beam shaping assembly, and are shown as follows:

Epithermal neutron flux>1×10⁹ n/cm²s

Fast neutron contamination<2×10⁻¹³ Gy-cm²/n

Photon contamination<2×10⁻¹³ Gy-cm²/n

Thermal to epithermal neutron flux ratio<0.05

Epithermal neutron current to flux ratio>0.7

Note: the epithermal neutron energy range is between 0.5 eV and 40 keV, the thermal neutron energy range is lower than 0.5 eV, and the fast neutron energy range is higher than 40 keV

1. Epithermal Neutron Flux

The epithermal neutron flux and the concentration of the boronated pharmaceuticals at the tumor site codetermine clinical therapy time. If the boronated pharmaceuticals at the tumor site are high enough in concentration, the epithermal neutron flux may be reduced. On the contrary, if the concentration of the boronated pharmaceuticals in the tumors is at a low level, it is required that the epithermal neutrons in the high epithermal neutron flux should provide enough doses to the tumors. The given standard on the epithermal neutron flux from IAEA is more than 109 epithermal neutrons per square centimeter per second. In this flux of neutron beams, therapy time may be approximately controlled shorter than an hour with the boronated pharmaceuticals. Thus, except that patients are well positioned and feel more comfortable in shorter therapy time, and limited residence time of the boronated pharmaceuticals in the tumors may be effectively utilized.

2. Fast Neutron Contamination

Unnecessary dose on the normal tissue produced by fast neutrons are considered as contamination. The dose exhibit positive correlation to neutron energy, hence, the quantity of the fast neutrons in the neutron beams should be reduced to the greatest extent. Dose of the fast neutrons per unit epithermal neutron flux is defined as the fast neutron contamination, and according to IAEA, it is supposed to be less than 2*10⁻¹³ Gy-cm²/n.

3. Photon Contamination (Gamma-Ray Contamination)

Gamma-ray long-range penetration radiation will selectively result in dose deposit of all tissues in beam paths, so that lowering the quantity of gamma-ray is also the exclusive requirement in neutron beam design. Gamma-ray dose accompanied per unit epithermal neutron flux is defined as gamma-ray contamination which is suggested being less than 2*10⁻¹³ Gy-cm²/n according to IAEA.

4. Thermal to Epithermal Neutron Flux Ratio

The thermal neutrons are so fast in rate of decay and poor in penetration that they leave most of energy in skin tissue after entering the body. Except for skin tumors like melanocytoma, the thermal neutrons serve as neutron sources of BNCT, in other cases like brain tumors, the quantity of the thermal neutrons has to be lowered. The thermal to epithermal neutron flux ratio is recommended at lower than 0.05 in accordance with IAEA.

5. Epithermal Neutron Current to Flux Ratio

The epithermal neutron current to flux ratio stands for beam direction, the higher the ratio is, the better the forward direction of the neutron beams is, and the neutron beams in the better forward direction may reduce dose surrounding the normal tissue resulted from neutron scattering. In addition, treatable depth as well as positioning posture is improved. The epithermal neutron current to flux ratio is better of larger than 0.7 according to IAEA.

The prosthesis beam quality factors are deduced by virtue of the dose distribution in the tissue obtained by the prosthesis according to a dose-depth curve of the normal tissue and the tumors. The three parameters as follows may be used for comparing different neutron beam therapy effects.

1. Advantage Depth

Tumor dose is equal to the depth of the maximum dose of the normal tissue. Dose of the tumor cells at a position behind the depth is less than the maximum dose of the normal tissue, that is, boron neutron capture loses its advantages. The advantage depth indicates penetrability of neutron beams. Calculated in cm, the larger the advantage depth is, the larger the treatable tumor depth is.

2. Advantage Depth Dose Rate

The advantage depth dose rate is the tumor dose rate of the advantage depth and also equal to the maximum dose rate of the normal tissue. It may have effects on length of the therapy time as the total dose on the normal tissue is a factor capable of influencing the total dose given to the tumors. The higher it is, the shorter the irradiation time for giving a certain dose on the tumors is, calculated by cGy/mA-min.

3. Advantage Ratio

The average dose ratio received by the tumors and the normal tissue from the brain surface to the advantage depth is called as advantage ratio. The average ratio may be calculated using dose-depth curvilinear integral. The higher the advantage ratio is, the better the therapy effect of the neutron beams is.

To provide comparison reference to design of the beam shaping assembly, we also provide the following parameters for evaluating expression advantages and disadvantages of the neutron beams in the embodiments of the present disclosure except the air beam quality factors of IAEA and the abovementioned parameters.

1. Irradiation time≤30 min (proton current for accelerator is 10 mA)

2. 30.0RBE-Gy treatable depth≥7 cm

3. The maximum tumor dose≥60.0RBE-Gy

4. The maximum dose of normal brain tissue≤12.5RBE-Gy

5. The maximum skin dose≤11.0RBE-Gy

Note: RBE stands for relative biological effectiveness. Since photons and neutrons express different biological effectiveness, the dose above should be multiplied with RBE of different tissues to obtain equivalent dose.

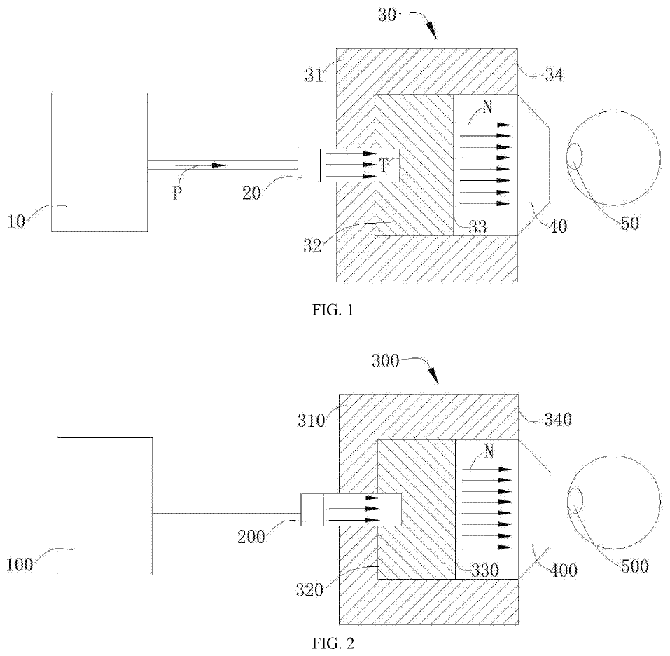

Please refer to FIG. 1, which shows a schematic plan view of an accelerator-based boron neutron capture therapy system, the boron neutron capture therapy system includes an accelerator 10, a beam expander 20, a charged particle beam inlet for passing the charged particle beam P, a charged particle beam P, a neutron generator T for generating a neutron beam N by nuclear reaction with the charged particle beam P, a beam shaping assembly 30 for adjusting the neutron beam flux and quality generated by the neutron generator T, a collimator 40 adjacent to the beam shaping assembly 30, and an α-amino acid-like boron trifluoride compound 50 irradiated by a beam emerging from the collimator 40. Wherein, the accelerator 10 is used to accelerate the charged particle beam P and may be an accelerator suitable for an accelerator type neutron capture therapy system such as a cyclotron or a linear accelerator; the charged particle beam P herein is preferably a proton beam; the beam expander 20 is disposed between the accelerator 10 and the neutron generator T; the charged particle beam inlet is adjacent to the neutron generator T and accommodated in the beam shaping assembly 30, and the three arrows between the neutron generator T and the beam expander 20 serve as charged particle beam inlet; the neutron generator T is accommodated in the beam shaping assembly 30, and the neutron generator T herein is preferably lithium. The beam shaping assembly 30 includes a reflector 31, a moderator 32 surrounded by the reflector 31 and adjacent to the neutron generator T, a thermal neutron absorber 33 adjacent to the moderator 32, and a radiation shield 34 arranged in the beam shaping assembly 30. The neutron generator T generates the neutron beam N by a nuclear reaction with the charged particle beam P incident from the charged particle beam inlet, the moderator 32 moderates neutrons generated from the neutron generator T to an epithermal neutron energy region, the reflector 31 directs the deviated neutrons back to increase the intensity of the epithermal neutron beam, the thermal neutron absorber 33 is used to absorb thermal neutrons to avoid overdosing in superficial normal tissues during therapy, and the radiation shield 34 is used to shield leaking neutrons and photons to reduce dose of the normal tissue not exposed to irradiation. A collimator 40 is used to converge the neutron beam; and the energy generated from the action of the neutron beam emitted through the collimator 40 on the α-amino acid-like boron trifluoride compound 50 destroys tumor cell DNA.

Please refer to FIG. 2, which shows a schematic plan view of a reactor-based boron neutron capture therapy system, the boron neutron capture therapy system includes a reactor 100 (a neutron beam is generated in the reactor, so it can also be called neutron generator), beam expander 200, a neutron beam inlet, a beam shaping assembly 300 for adjusting the neutron beam flux and quality generated by the neutron generator, a collimator 400 adjacent to beam shaping assembly 300, and an α-amino acid-like boron trifluoride compound 500 irradiated by a beam emerging from the collimator 40. Wherein, the reactor 100 may be a related nuclear reaction capable of generating neutron with required energy, which is well known to those skilled in the art, such as fast neutrons generated from fission reactions of uranium-235 or plutonium-239; the beam expander 200 is disposed between the reactor 100 and the neutron beam inlet; and the three arrows following the beam expander 200 serve as neutron beam inlets. The beam shaping assembly 300 includes a reflector 310, a moderator 320 surrounded by the reflector 310, a thermal neutron absorber 330 adjacent to the moderator 320, and a radiation shield 340 arranged in the beam shaping assembly 300. The moderator 32 moderates neutrons generated from the neutron generator 100 to an epithermal neutron energy zone, the reflector 310 directs the deviated neutrons back to increase the intensity of the epithermal neutron beam, the thermal neutron absorber 330 is used to absorb thermal neutrons to avoid overdosing in superficial normal tissues during therapy, and the radiation shield 340 is used to shield leaking neutrons and photons to reduce dose of the normal tissue not exposed to irradiation. The collimator 400 is used to converge the neutron beam; and the energy generated from the action of the neutron beam emitted through the collimator 400 on the α-amino acid-like boron trifluoride compound 500 destroys tumor cell DNA.

The beam shaping assembly 30, 300 can moderate the neutrons to the epithermal neutron energy region and reduce the thermal neutron and fast neutron content. The reflectors 31, 310 are made of a material having strong neutron reflection capability. As a preferred embodiment, the reflectors 31, 310 are made of at least one of Pb or Ni. The moderators 32, 320 are made of a material having a large action cross section for fast neutron and a small action cross section for epithermal neutron. As a preferred embodiment, the moderators 32, 320 are made of at least one of D₂O, AlF₃, Fluental™, CaF₂, Li₂CO₃, MgF₂ and Al₂O₃. The thermal neutron absorbers 33, 330 are made of a material having a large action cross section for thermal neutron. As a preferred embodiment, the thermal neutron absorbers 33, 330 are made of ⁶Li. The radiation shields 34, 340 include photon shield and neutron shield. As a preferred embodiment, the radiation shields 34, 340 include a photon shield made of Pb and a neutron shield made of polyethylene (PE). The collimators 40, 400 are made of a material having a strong neutron gathering capability. As a preferred embodiment, the collimators 40, 400 are made of at least one of graphite and Pb.

Those skilled in the art know that, besides the above accelerator type and reactor type neutron generation methods, other neutron generation methods, such as D-D neutron generator, D-T neutron generator, etc., can also be used according to actual needs. The material, structure, and composition of the beam shaping assembly can also be adjusted according to actual needs.

In BNCT, when administered in a therapeutically effective amount, the boron-containing compound must be non-toxic or low toxic, and can selectively accumulate in tumor tissue. Although BPA has the advantage of low chemical toxicity, it accumulates in critical normal tissues at below-desired levels. In particular, the ratio of boron in tumor relative to normal brain and tumor to blood is approximately 3:1. This low specificity limits the maximum dose of BPA to the tumor because the allowable dose for normal tissue is a limiting factor.

Therefore, there is a need to develop new compounds that have a longer retention time in tumors and selectively target and destroy tumor cells with minimal damage to normal tissues.

α-amino acids are the main components of proteins and are the most important amino acids in organisms. They play a very important role in the production of ATP and in the process of neurotransmission. In addition, α-amino acids are also key nutrients for the survival and proliferation of cancer cells. α-Amino acid-like boron trifluoride compound are obtained by replacing —COOH in the α-amino acid with —BF₃, which is an α-amino acid isoelectronic compound. Studies have shown that cellular pathways for ingesting α-amino acid-like boron trifluoride compounds are the same as α-amino acids, and they all adopt enzyme-mediated pathways, and both have the same transporter. The α-amino acid-like boron trifluoride compounds have attracted our intense attention in the design of novel boron carrier compounds for BNCT, which have high stability, good targeting, and high enrichment in tumor cells. Compared to FDG, the uptake of these compounds by the inflammatory region is almost negligible. In addition, the α-amino acid-like boron trifluoride compounds are readily synthesized and are usually prepared by reacting the corresponding boronic acid ester with KHF₂ under acidic conditions.

In addition, using ¹⁸F-labeled α-amino acid-like boron trifluoride compounds in BNCT, boron concentrations and distributions in and around tumors and all tissues within the radiation therapy volume can be measured non-invasively and accurately before and during irradiation. This diagnostic information enables boron neutron capture therapy to be performed faster, more accurately, and more safely by reducing the exposure of epithermal neutrons to tissue regions known to contain high levels of boron.

The α-amino acid-like boron trifluoride compound is described in details below in connection with specific examples.

### Example 1 Preparation of Phe-BF₃

Preparation Route:

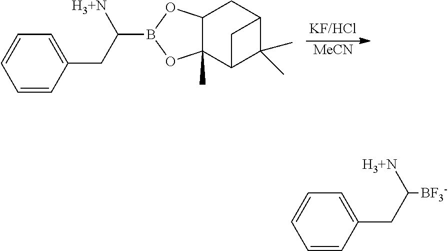

Benzyl borate (15 mg, 0.05 mmol), KF (0.15 mmol, 0.05 mL) solution, HCl (0.2 mmol, 0.03 mL) solution, 0.1 mL MeCN solution were added to a 1.5 mL microreactor and reacted for 2 h at room temperature to give crude Phe-BF₃. The crude product was further purified by HPLC to give Phe-BF₃. ¹H NMR (300 MHz, MeOD): δppm 7.30 (m, 5H), 3.04 (d, J=9.8 Hz, 1H), 2.67 (t, J=9.8 Hz, 1H), 2.42 (brs, 1H); [M-H]⁻ 188.0901, Found: 188.05890.

In Vitro Study of the Compound According to the Disclosure

An in vitro test of the Phe-BF₃ of the Example 1 uses four different human-derived tumor cell lines U343mga, human liver cancer cell line Hep3B, human breast cancer cell line MCF7 and human Sarcoma cell line 4SS. Cells were plated on uncoated tissue culture dishes and incubated at 37° C. in an incubator with humidified air equilibrated with 5% CO₂ (the medium was supplemented with 10% FCS and PEST (Penicillin 100 IU/mL and streptomycin 100 mg/mL)). For cell passage, cells were trypsinized with trypsin-EDTA (phosphate buffered saline (PBS) with 0.25% trypsin and 0.02% EDTA, no calcium and magnesium).

### Example 2 Cell Uptake of Phe-BF₃

U343mga cells were plated on Petri dishes at a cell density of 75% and incubated for 6 hours with 1,4-dihydroxyborylphenylalanine (BPA) or Phe-BF₃ dissolved in tissue culture medium. Both boron-containing compounds were added at an equimolar concentration relative to the boron content (5×10⁻⁴ mol/L boron) and dissolved in tissue culture medium. Incubation was terminated by removing the boron containing tissue culture medium and adding cold phosphate buffered saline solution (PBS buffer) in order to wash away excess medium from the cells. Cells were immediately harvested by scooping off from the petri dishes using rubber policeman, and collected in cold PBS and pelleted by centrifugation.

Total protein analysis was performed on cell samples according to the Bradford standard procedure. The precipitated cells were subjected to boron analysis by DC-plasmon atomic emission spectroscopy (DCP-AES). The sample (50-130 mg) was digested with sulfuric acid/nitric acid (1/1) at 60° C. Triton X-100 and water were added to give concentrations of 50 mg tissue/mL, 15% total acid v/v, and 5% Triton X-100 v/v. The boron concentration is based on known control sample. The results are shown in Table 1 below. As can be seen from Table 1, Phe-BF₃ is better than boron phenylalanine (BPA) in boron uptake.

TABLE 1
Cell uptake of different boron compounds
For the different boron compounds in the two parallel experiments
(Trials 1 and 2), the boron content is expressed as a function
of the total cellular protein in U343mga cells (7.2 and 7.7 μg boron/mL
media in Trial 1 and Trial 2, respectively).

| Boron compound | Trial 1 | Trial 2 |
|---|---|---|
| BPA | 62 | 39 |
| Phe-BF₃ | 549 | 421 |

### Example 3 Uptake of Phe-BF₃ by Different Tumor Cells

Four human-derived, different tumor cell lines: U343mga, Hep3B, MCF7, and 4SS were plated on Petri dishes at 40-50% (low) and 90-100% (high) cell densities, and incubated with Phe-BF₃ in tissue culture as described above for 6 hours. Incubation was terminated by removing the boron-containing media and adding cold PBS buffer to wash excess media from the cells. Cells were immediately harvested by scooping off from the petri dishes using rubber policeman, and collected in cold PBS and pelleted by centrifugation. Total protein analysis was performed on cell samples according to the Bradford standard procedure (as described above). The results are shown in Table 2 below. From a comparison of all four human tumor cell lines tested at low and high cell densities, Phe-BF₃ was found to be a highly efficient boron carrier.

TABLE 2
Cell uptake of Phe-BF₃. The boron content is expressed as a function
of the total cellular protein (μg boron/g cell protein).

| Cell line | Low cell density | High cell density |
|---|---|---|
| U343mga | 102 | 178 |
| Hep3B | 149 | 207 |
| MCF7 | 101 | 167 |
| 4SS | 255 | 343 |

### Example 4 Intracellular Retention of Phe-BF₃

U343mga cells were plated on Petri dishes at a cell density of 75% and incubated for 18 hours with 1,4-dihydroxyboron-phenylalanine (BPA) or Phe-BF₃ in tissue culture medium. Both boron compounds were added to the tissue culture medium at equimolar concentrations relative to the boron content (5×10⁻⁴ mol/L boron). The incubation was terminated by replacing the boron-containing medium with a boron-free medium. Cell samples were taken at time points 0, 2 and 7 hours, respectively, where the 0 time point represented the time point when the incubation with the boron compound reached just 18 hours.

The cells were washed with cold PBS and immediately harvested by scooping off from the petri dish using rubber policeman, and collected in cold PBS and pelleted by centrifugation. The cell pellets were analyzed for total protein and boron content as described above. The results are shown in Table 3 below. With intracellular uptake, the compound of formula (I) retained in the tumor cells was 50% of the total uptake at 7 h after the complete consumption of Ia in the medium.

TABLE 3
Boron content (μg boron/g cell pellet) in
U343mga cells at 0, 2 and 7
h after removal of the boron-containing medium.

| Boron compound | 0 h | 2 h | 7 h |
|---|---|---|---|
| BPA | 0.072 | 0 | 0 |
| Ia | 0.83 | 0.56 | 0.41 |

In summary, as shown in Examples 2-4, the compound Phe-BF₃ has shown the expected results in an in vitro assay, which is superior to BPA in tumor cell uptake, accumulation, and retention.

### Example 5 Phe-BF₃ Cytotoxicity Study Test

The cell culture fluid containing the peptide bovine serum was incubated at 37 C for 24 hours. The passage cultured mouse fibroblast L-929 cells were used to prepare a cell suspension of 1×10⁵ cells/mL, and the cell suspension was seeded on a 96-well cell culture plate (100 μl/well) and cultured in a carbon dioxide incubator at 37° C. for 24 hours. After the cells adhere to the wall, the supernatant was removed, the control solution (in absence of compound Ia) was added, and the culture solution of the test group (Phe-BF₃ concentration of 5 mmol/L) was exchanged, and the culture was continued in the carbon dioxide incubator at 37° C. After 2 days, MTT solution was added to continue culture for 4 hours. The original solution was aspirated and DMSO was added and shaken for 10 min. The absorbance value was measured with an enzyme-linked immunometric instrument at a wavelength of 630 nm, and the relative proliferation rate (RGR) of the cells was calculated according to the absorbance according to the formula. The results are shown in Table 4 below.

TABLE 4
Relative cell proliferation (RGR) results measured by MTT
colorimetry

| NO. | Time (days) | RGR (%) |
|---|---|---|
| 1 | 2 | 123 |
| 2 | 7 | 102 |

$\begin{matrix}Note \text{:} \\ RGR ( \% ) = \frac{average absorbance of test group}{average absorbance of control group} \times 100 \% .\end{matrix}$

Cellular toxicity was assessed based on cell relative proliferation, shown in Table 5 below.

TABLE 5
Assessment of cytotoxicity

| Grade | Relative proliferation RGR (%) |
|---|---|
| 0 | ≥100 |
| 1 | 75~99 |
| 2 | 50~74 |
| 3 | 25~49 |
| 4 | 1~24 |
| 5 | 0 |

As can be seen from Table 5, Phe-BF₃ showed no signs of toxicity.

The boron neutron capture therapy system disclosed in the present disclosure is not limited to the contents described in the above embodiments and the structures shown in the drawings. Apparent changes, substitutions, or modifications in the present disclosure are to be understood as being included within the scope of the present invention as defined by the appended claims.

What is claimed is:

1. A boron neutron capture therapy system comprising:
a boron neutron capture therapy device; and
an α-amino acid-like boron trifluoride compound having a structure shown as formula (I) below: 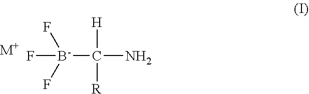
wherein, R represents hydrogen, methyl, isopropyl, 1-methylpropyl, 2-methylpropyl, hydroxymethyl, 1-hydroxyethyl, benzyl or hydroxybenzyl;
M represents H or metal atom;
the energy generated from the action of the neutron beam generated by the boron neutron capture therapy device on the α-amino acid-like boron trifluoride compound destroys tumor cell DNA.

2. The boron neutron capture therapy system according to claim 1, wherein the boron neutron capture therapy device comprises a neutron generator and a beam shaping assembly for adjusting the neutron beam energy spectrum generated by the neutron generator to an epithermal neutron energy zone.

3. The boron neutron capture therapy system according to claim 2, wherein the beam shaping assembly comprises:
a moderator adjacent to the neutron generator, wherein the moderator moderates neutrons generated from the neutron generator to an epithermal neutron energy zone,
a reflector surrounding the moderator, wherein the reflector leads the deflected neutrons back to increase the intensity of an epithermal neutron beam,
a thermal neutron absorber adjacent to the moderator, wherein the thermal neutron absorber absorbs thermal neutrons to avoid overdosing in superficial normal tissues during therapy, and
a radiation shield arranged in the beam shaping assembly, wherein the radiation shield shields leaking neutrons and photons to reduce dose of the normal tissue not exposed to irradiation.

4. The boron neutron capture therapy system according to claim 3, wherein the boron neutron capture therapy device further comprises a collimator disposed at the beam outlet for converging the epithermal neutrons.

5. The boron neutron capture therapy system according to claim 1, wherein M represents potassium or sodium.

6. The boron neutron capture therapy system according to claim 1, wherein the element B is ¹⁰B.

7. The boron neutron capture therapy system according to claim 6, wherein the purity of ¹⁰B in the α-amino acid-like boron trifluoride compound is ≥95%.

8. The boron neutron capture therapy system according to claim 1, wherein at least one element F in the α-amino acid-like boron trifluoride compound is ¹⁸F.
